(19) United States
(12) Patent Application Publication
(10) Pub. No.: US 2011/0184382 A1
(43) Pub. Date: Jul. 28, 2011

(54) MULTI-PURPOSE ARTICLES FOR SANITIZING AND CAPPING LUER ACCESS VALVES

(76) Inventor: Timothy B. CADY, Encinitas, CA (US)

(21) Appl. No.: 12/860,822

(22) Filed: Aug. 20, 2010

Related U.S. Application Data
(60) Provisional application No. 61/235,659, filed on Aug. 20, 2009.

(51) Int. Cl. A61M 5/31; A61L 2/18

(52) U.S. Cl. 604/506; 604/256; 422/28

(57) ABSTRACT

Multi-purpose devices for capping fluid reservoirs and sanitizing accessible surfaces of fluid-transporting medical fittings (e.g., luer access valves) at risk of contamination with infectious agents are described, as are methods for making and using such devices.

### RELATED APPLICATION

This application claims the benefit of and priority to provisional application Ser. No. 61/235,659 (Attorney docket no. ZNC-1020-PV), filed on 20 Aug. 2009, the contents of which are herein incorporated by reference in their entirety for any and all purposes.

### TECHNICAL FIELD

This invention concerns small disposable devices useful for both sanitizing luer access valves and capping fluid delivery devices such as syringes.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Exposure to infectious agents (e.g., pathogenic bacteria, viruses, fungi, etc.) in medical settings is a matter of serious concern. One route of exposure to such agents is the opening made in skin by the bore of needle, canula, or other similar device used to provide access to a patient's vasculature. It is known that patients whose skin has been compromised in this way are at increased risk for developing serious blood stream infections. In the United States alone, approximately 300,000 blood stream infections per year result from the installation and use of peripheral intravenous catheters (PIVC), and more than 80,000 blood stream infections are associated with the use central venous catheters (CVC). All told, in the U.S. approximately 20,000 patients die annually from hospital acquired infections that result from PIVC and CVC use. Costs associated with the care and treatment of patients that develop infections due to PIVC and CVC use exceed $2.7 billion.

In hospital settings today, occupational health and safety regulations designed reduce the risk to health care workers from needle prick and similar injuries have resulted in the deployment of needleless medical valves whenever possible. Currently, more than 500 million needleless valves, also known as luer access valves, are used annually in hospitals throughout the U.S. Needleless valves are used primarily in conjunction with PIVC and CVC devices, which may contain from as few as one to as many as 3, 4, 5, or more luer access valves (LAVs).

The widespread use of needleless valves in acute medicine has contributed to a marked increase in the incidence of hospital acquired infections, particularly blood stream infections. To reduce the risk of infection from a contaminated luer access valve, standard practice today requires that a nurse or other health care worker clean the exposed or accessible surfaces of a luer access valve by rubbing it with a sterile alcohol swab or wipe immediately prior to making a connection to the LAV, for example, by attaching a syringe or intravenous (IV) set to the LAV to deliver a medication via a PIVC or CVC already connected to a patient.

Other approaches have also been suggested, such as placing caps on each luer access valve when it is not being accessed. Of course, after a particular LAV is used, it must then be recapped with a new cap. Such approaches are expensive and time-consuming and thus will likely be impractical in clinical settings.

More recently, an innovative solution has appeared, and is thoroughly described in commonly owned U.S. non-provisional parent application Ser. Nos. 12/143,787 and 12/538,556, filed 21 Jun. 2008 and 11 Aug. 2009, which applications, as well as the two now-expired U.S. provisional patent applications from which each of these non-provisional applications claims priority (U.S. Ser. No. 60/945,696 and 60/979,819, filed 22 Jun. and 13 Oct. 2007, respectively), are hereby incorporated in their entirety for all purposes. Briefly, that solution concerned a variety of patentable, single-use sanitizing devices that can be used to effectively and efficiently sanitize, and preferably sterilize, exposed surfaces of LAVs, particularly the accessible surface of the valve stems of LAVs, particularly those surfaces that may become contaminated with infectious or pathogenic agents. Such devices generally comprise a sanitizing element integrated within a shell. A sanitizing element comprises a substrate and a sanitizing reagent dispersed in the substrate prior to use, preferably at the time the device is manufactured. The sanitizing element substrate includes a sanitizing region capable of engaging an accessible surface of a valve stem of a LAV so as to expose the accessible surface, and any infectious agents residing thereon, to the sanitizing reagent. Despite such advances, however, there remains a need for additional solutions to address the problem of potential contamination of the multitude of connectors, fittings, and other devices utilized in clinical settings in conjunction with administering various fluids and medicines to patients.

3. Definitions

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

An “aqueous solution” refers to a water-based solution capable of dissolving or dispersing one or more other substances, or solutes (i.e., the substance(s) dissolved in the solvent). A “solution” is a homogeneous mixture of at least one substance in a liquid. In the context of this invention, “aqueous solvents” can also include other liquids, including organic liquids, such as alcohols and/or oils.

An “infectious agent” refers to any organism capable of infecting another organism. Such agents include many bacteria, viruses, and fungi.

A “patentable” composition, process, machine, or article of manufacture according to the invention means that the subject matter at issue satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically excludes the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

In a “suspension” solid particles are dispersed in a liquid. The term “colloidal” refers to a state of subdivision, which, in the context of solutions, means that molecules or particles dispersed in the liquid have at least in one direction a dimension roughly between 1 nm and 1 μm. It is not necessary for all three dimensions to be in the colloidal range. A “colloidal dispersion” is a system in which particles of colloidal size of any nature (e.g. solid, liquid or gas) are dispersed in a continuous phase of a different composition (or state). In an “emulsion” liquid droplets and/or liquid crystals are dispersed in another liquid. An emulsion may be denoted by the symbol “O/W” if the continuous phase (i.e., is an aqueous solution) and by “W/O” if the continuous phase is an organic liquid.

### SUMMARY OF THE INVENTION

It is an object of this invention to provide patentable multi-function devices suitable for both sanitizing, and preferably sterilizing, exposed surfaces of luer access valves, particularly those surfaces that may become contaminated with infectious agents, and capping the fluid delivery end (i.e., discharge port) of medical fluid reservoir (e.g., an IV bag, a fluid-filled syringe, etc.) in order to prevent contamination of fluid delivery portion of the fluid reservoir. In the context of the invention, “sanitize” encompasses cleaning, disinfecting, and/or sterilizing. Such devices can incorporate these functions in an integrated, single piece device; alternatively, multi-part devices can be used.

The features and advantages of the invention will be apparent from the following drawings, detailed description, and appended claims.

### BRIEF DESCRIPTION OF THE FIGURES

FIG. 1 shows a preferred 2-piece embodiment of the invention. In the figure, the two pieces, A and B, of the capping/sanitizing article are shown disconnected from each other. Also shown is a luer access valve, C, that can be sanitized using the sanitizing component of piece B and capped using LAV-capping component of piece A.

FIG. 2 depicts an embodiment of the invention wherein the capping/sanitizing article (10) is attached to a fluid-filled syringe (20). As shown, the sanitizing component still retains its removable seal (11). Also shown is a luer access valve (C).

### DETAILED DESCRIPTION

As those in the art will appreciate, the following detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

This invention concerns patentable multi-purpose articles that can be used to (1) effectively and efficiently clean, disinfect, and preferably sterilize, exposed surfaces of luer access valves such as needleless medical valves, as these surfaces are at risk for contamination with infectious agents such as bacteria, fungi, and viruses, and (2) cap the fluid-dispensing end, particularly the discharge port, of a fluid reservoir such as a fluid-filled syringe or luer connector of an IV set.

“Multi-use” (or “multi purpose”) refers to an article or device suitable for two or more uses or purposes, as distinguished from “single” use or purpose devices. Thus, in the context of the invention, a “multi-purpose” article or device is one that has a component or portion useful for sanitizing, for example, a LAV, and another component or portion configured to cap the discharge port of medical fluid reservoir (e.g., a pre-filled syringe, the luer connector of an IV set, etc.). In some embodiments, the article of the invention also includes a third component or portion configured to cap that part of a LAV that has just been sanitized using the sanitizing component of the article. As those in the art will appreciate, after each component has been used, it typically will not be suitable for any further use or purpose. In other words, a sanitizing component would typically be used to sanitize one LAV, a cap for a discharge port would not be used to cap any other device, and a cap for LAV would be used once, such that after its removal another cap would preferably (although not necessarily) be used to recap the LAV, depending on the configuration of the particular article.

In general, the reservoir-capping/LAV-sanitizing articles of the invention each comprise a sanitizing component that includes a sanitizing element disposed in a shell such that the sanitizing element can be maintained in a clean, preferably sterile, condition until it is used to sanitize (i.e., clean, disinfect, or sterilize) a luer access valve (LAV), such as a needleless medical valve. The article also includes a medical fluid reservoir cap component, and in some embodiments, at least one additional cap component, particularly a LAV cap.

Herein, a sanitizing element comprises a sanitizing reagent dispersed in a substrate. In some embodiments, the sanitizing reagent is dispersed in or otherwise combined with the substrate during the process used to manufacture the sanitizing element, while in other embodiments, the device is configured such that the sanitizing reagent is released for dispersion into the substrate post-manufacture, either before at the time the device is brought into contact with the LAV to be sanitized.

In accordance with the invention, a sanitizing reagent comprises an active ingredient capable of sanitizing a surface of a needleless medical valve. Any active ingredient that can be used effectively to rapidly sanitize a LAV can be adapted for use in practicing the invention, and are generally classified as antibacterial and antifungal agents, antiseptic or antimicrobial agents, wide spectrum disinfectants, and/or parasiticides, as well as combinations of such reagents. Particularly preferred are biocompatible active ingredients and sanitizing reagents, as the devices of the invention are intended for human and/or veterinary use, including alcohols, antibiotics, oxidizing agents, and metal salts. Representative examples of such active ingredients include bleach, chlorhexidine, ethanol, isopropyl alcohol, hydrogen peroxide, sodium hydroxide, and an iodophor dissolved or otherwise dispersed in a suitable solution, suspension, or emulsion. Other active ingredients having suitable sanitizing effects can also be used. These include alcohols (e.g., ethanol, benzyl alcohol, isopropyl alcohol, phenoxyethanol, phenethyl alcohol, etc.); antibiotics (e.g., aminoglycosides, such as amikacin, apramycin, gentamicin, kanamycin, neomycin, netilmicin, paromomycin, rhodostreptomycin, streptomycin, and tobramycin; bacitracin; chloramphenicol; erythromycin; minocycline/rifampin; tetracycline; quinolones such as oxolinic acid, norfloxacin, nalidixic acid, pefloxacin, enoxacin and ciprofloxacin; penicillins such as oxacillin and pipracil; nonoxynol 9; fusidic acid; cephalosporins; etc.), quaternary ammonium chlorides; quaternary ammonium carbonates; benzalkonium chloride; chlorinated phenols; fatty acid monoesters of glycerin and propylene glycol; iodine; iodine containing compounds, such as 3-iodo-2-propynyl butyl carbamate (IPBC); iodophors, such as povidone-iodine (Betadine 100%, which contains providine iodine as the active ingredient); hydantoins, such as dimethylhydantoin and halogenated hydantoins; isothiazolinones; parabens, such as methylparaben, ethylparaben, and propylparaben; chloroxylenol; chlorhexidine and its salts; chlorhexidine/silver-sulfadiazine; chlorhexidine acetate; chlorhexidine gluconate (e.g., Hibiclens); chlorhexidine hydrochloride; chlorhexidine sulfate; benzoic acid and salts thereof; benzalkonium chloride; benzethonium chloride; methylbenzethonium chloride; chlorobutanol; sorbic acid and salts thereof; imidazole antifungals (e.g., miconazole); butocouazole nitrate; mafenide acetate; nitrofurazone; nitromersol; triclocarban; phenylmercuric nitrate or acetate (0.002%); chlorocresol; chlorbutol; clindamycin; CAE (Anjinomoto Co., Inc., containing DL-pyrrolidone carboxylic acid salt of L-cocoyl arginine ethyl ester); cetylpyridinium chloride (CPC) at 0.2%, 0.02%, and 0.002% concentrations; 9.8% isopropyl alcohol; 1% ZnEDTA; mupirocin; and polymyxin (polymyxin b sulfate-bacitracin). Additionally, other useful compounds and compositions include Miconazole, Econazole, Ketoconazole, Oxiconizole, Haloprogin, Clotrimazole, butenafine HCl, Naftifine, Rifampicin, Terbinafine, Ciclopirox, Tolnaftate, Lindane, Lamisil, Fluconazole, Amphotericin B, Ciprofloxecin, Octenidine, Triclosan (2,4,4′-trichloro-2′-hydroxydiphenyl ether), Microban (5-chloro-2-phenol (2,4 dichlorophenoxy). Useful metal-based sanitizing reagents include silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine.

The particular active ingredient(s) selected as a sanitizing reagent for a given application will be compatible with the sanitizing element substrate and material(s) used to form the shell of the particular article. In some embodiments, the sanitizing reagent is dispersed in the substrate after the substrate is formed, for example, by saturating or supersaturating the substrate material with the sanitizing reagent before or after it is coated or integrated with a pre-fabricated housing. In other embodiments, it is dispersed during the process used to manufacture the substrate. In still other embodiments, the article is configured such that the sanitizing reagent is released from a rupturable or frangible reservoir adjacent to the substrate when the sanitizing component is brought into contact with the LAV to be sanitized.

As will be appreciated, the materials used to prepare the sanitizing reagent should be compatible with the constituent or constituents that comprise the substrate such that the substrate does appreciably degrade or otherwise suffer loss of structural integrity prior to being used to sanitize a medical valve or region of a patient's skin. Similarly, the sanitizing reagent should be biocompatible, such that it will not harm a patient's skin the event of contact or should some amount of the sanitizing reagent inadvertently be admitted into the fluid carrying portion of a needleless medical valve, as well as with materials used to form needleless medical valves.

In preferred embodiments, the substrate used to form a sanitizing element is any suitable absorbent, pliable, fibrous, or porous material, or combination of materials, than can be wetted and/or impregnated with a sanitizing reagent. Such materials include those that are synthetic or naturally occurring, and they may be of homogeneous or heterogeneous composition. Preferred synthetic materials include fibrous, foam, and gel compositions, particularly those having directionally oriented natural or synthetic fibers, or combinations thereof. Preferred naturally occurring materials useful as substrates include fibrous naturally occurring materials, including plant-derived materials such as cotton and paper products, as well as animal-based fiber products such as wool. Other preferred natural materials are sponges.

As will be appreciated, in order to achieve the desired sanitizing effect, a sanitizing element, or the component part(s) thereof designed to contact a LAV, preferably are made of a material (or combination of materials) that allow the sanitizing element to thoroughly sanitize exposed surfaces of LAVs, particularly those surfaces that are exposed to air, touch, or other contact and thus are at risk for contamination with infectious agents, and are also intended to form part of the fluid flow path for fluids to be introduced into a patient, for example, IV solutions, medications, blood and blood products, etc. Preferably, the substrate material should be sufficiently compliant to allow that portion of a LAV that contains the fluid access port to be associated with, and in preferred device configurations, inserted into an article according to the invention, yet conform to the three-dimensional external configuration of the LAV to assure intimate contact to at least those exposed surfaces of the valve intended to come into contact with fluid. In addition, the substrate allows for the retention of a liquid sanitizing reagent, for example, in capillary spaces, in the void volume of sponges, etc. The substrate may also be formulated such that its surface is modified to include sanitizing reagents such as silver ions and/or other suitable materials.

A particularly preferred class of materials for substrate fabrication is directionally oriented fibrous materials. These include, without limitation, materials comprised of cellulose fibers, glass fibers, and polyester fibers, as well as materials comprised of combinations of two of more of these and/or other materials. A particularly preferred fibrous substrate material is that used to form Transorb XPE® reservoirs (Filtrona Fibertec, Richmond, Va.). Such bonded synthetic fibers use capillary action to precisely absorb, retain, transfer, and/or release liquids or vapor in desired amounts. A broad range of synthetic polymers can be used to form the fibers, and, if desired, they may be treated for functional purposes, for example, to contain a sanitizing reagent dispersed therein, to provide a vapor barrier or other coating over a portion of the product's surface, etc. The geometric shape of these materials can also be customized for particular applications, thereby permitting easy integration of the substrate into desired device forms. Furthermore, the materials can include chemicals to indicate a functional change in the substrate, for example, by using a color change to signal a change from a wet to a dry state. In this way, a color change in the substrate could be used to indicate that the substrate has dried out and should not be used, perhaps due to a leak in the article's storage container.

Other representative classes of materials suitable for use as substrates include gel-forming polymers such as agarose, agar, polyacrylamide, and other synthetic porous materials that can be formed into layers, sheets, columns, or other shapes compatible with practicing the invention. Representative gelatinous materials include hydrogels (i.e., cross-linked polymers that absorb and hold water), particularly those made from agarose, (2-hydroxyethyl)methacrylate and its derivatives, and synthetic carbohydrate acrylamides.

Still other classes of materials include porous polymer sponges. Such sponges can be formed from any suitable material, including polyethylene, polypropylene, olytetrafluoroethylene, polyvinylidine difluoride, polynitrile, and polystyrene. Many such porous polymer sponges are commercially available in a wide variety of shapes, pore density and size, etc. Additionally, conventional foam forming techniques can be used to make polymer sponges by polymerizing appropriate monomers. In general, sponges have an open pore structure to allow movement of a solvent such as a liquid sanitizing reagent. The sponge surface should include open pores to provide entry of liquid sanitizing reagents (e.g., alcohol, iodine-containing solutions, etc.), and, as with other materials used to form substrates, the particular substrate material chosen is preferably inert, i.e., not reactive with components of the sanitizing reagent, the shell of the article or its container, or the materials used to produce LAVs.

Surgical foams are another preferred class of substrate materials. The materials can be natural or synthetic, as desired. Suitable foams include rubber latex, polyurethane, polyethylene and vinyl foams. Preferably, such foams are made from any suitable biocompatible polymer, for example, polyvinyl alcohol (PVA) or polyurethane. One preferred foam material is Microbisan™, a hydrophilic polyurethane foam that is impregnated with silver ions (Lendell Manufacturing, St. Charles, Mich.). Preferably, such foams are highly absorbent and thus suitable for use with liquid sanitizing reagents. In other embodiments, the material used to form the foam is well-suited for dispersion of a dry sanitizing reagent, such as silver ions. Again, it is preferred that foam materials, if used to as a substrate, be inert. Also, they are preferably sufficiently flexible to conform to the variety of different shapes and surface configurations (e.g., double seal fluid access points, luer threads, etc.) encountered in the field given the multitude of luer access valve shapes, sizes, and configurations. In this way sufficient contact between the sanitizing surface(s) of the sanitizing element and the surface(s) of the medical valve to be cleansed can be ensured. Another advantage of some synthetic foams (as well as certain other polymeric materials from which substrates may be formed) is that they can easily be injected in a desired volume into a shell or housing during manufacture, after which they expand to assume the desired substrate size, density, porosity, etc.

Preferred natural materials include those derived from cotton and naturally occurring sponges. As those in the art appreciate, processed cotton fibers are composed almost entirely of the natural polymer cellulose. In such fibers, 20-30 layers of cellulose are coiled into a series of spring configurations, which makes the fibers absorbent and gives them a high degree of durability and strength. For example, woven cotton sheets, as are often used in the manufacture of sterile cleansing pads that are then saturated with a 70% isopropyl alcohol (IPA) solution, can be used as substrates. Any suitable configuration may be used. For example, a woven cotton sheet can be rolled to form a tube that can then be cut into small cylinders, before of after dispersing a suitable sanitizing reagent therein. In some embodiments of the invention, such cylinders can be used as substrates in the manufacture sanitizing elements that are then integrated with suitable shells or housings. Other fibers, be they naturally occurring, synthetic, or combinations of natural and synthetic materials, having similar properties can also readily be adapted for use as substrates to make sanitizing elements.

The sanitizing element of any substrate includes a sanitizing region capable of engaging an accessible surface of a valve stem of a needleless medical valve so as to expose the accessible surface, and any infectious agents residing thereon, to the sanitizing reagent. In many embodiments, the sanitizing region is the exposed, accessible surface (i.e., a sanitizing surface) of the sanitizing element designed to contact the surface to be sanitized, and the rest of the sanitizing element is inaccessible due to the shell or housing.

In some embodiments, an abrasive layer may be disposed on or comprises the upper surface of the substrate, such that the upper surface, or face, of the abrasive layer comes to for the sanitizing region of the sanitizing element. An abrasive layer typically is comprised of a natural or synthetic material, or combination of materials, that provide it with a greater abrasive or scrubbing capacity than material used to form the substrate, thereby enabling the abrasive layer to provide greater capacity to assist in the mechanical disruption or removal of biofilms (as, for example, may be formed by infectious agents contaminating the exposed surface(s) of needleless medical valves in a PIVC or CVC connected to a patient in a hospital or other healthcare setting) or other unwanted materials. It will also be understood that an “abrasive layer” can be formed in the upper portion of the substrate that includes the sanitizing region by a suitable treatment, such as heating, chemical treatment, and the like.

As already described, in some embodiments, the sanitizing element comprises a single layer, whereas in others, it comprises a plurality of layers. In multi-layer devices, the substrate used to form each layer can be of the same or different material, and may or may not contain a sanitizing reagent. Additionally, in some embodiments of multi-layer devices, one or more of the layers may be physically separated from the other layer(s) it contacts by an impermeable, semi-permeable, or permeable barrier.

For sanitizing elements that comprise multi-layered substrates, at least one of the layers contains a sanitizing reagent. In some such embodiments, each layer contains the same or a different sanitizing reagent. Here, a “different sanitizing reagent” means that each reagent contains either a different active ingredient(s), or the same active ingredient(s) in a different formulation or concentration. When different active ingredients are used, they are preferably compatible, such that one does not inactivate or otherwise degrade the sanitizing activity of the other active ingredient(s), nor should it materially degrade or chemically alter any substrate used to form a substrate layer or any material used to manufacture a medical fitting that can be sanitized by the device of the invention.

In embodiments wherein the sanitizing element is comprised of two or more layers, the substrate portion of each layer can be formed from a material that is the same as or different from the material used to form the substrate of one or more of the other layers, and each layer may contain the same, different, or even no, sanitizing reagent (although at least one layer will have a sanitizing reagent dispersed therein prior to engaging the surface of the needleless valve to be sanitized). Also, even when substrates for different layers are formed from the same material, they may be configured differently. For example, in a particularly preferred embodiment that employs a sanitizing element having two layers, where the substrate for each layer is formed from the same type of synthetic absorbent material having directionally fibers, the orientation of the fibers in one layer can differ from the fiber orientation in the other layer.

In any multi-purpose capping/sanitizing article according to the invention, the sanitizing element is encapsulated, enclosed, or housed in a suitable shell, housing, or other container or coating such that at least a portion of the sanitizing element, preferably its sanitizing region, is exposed for contact with a surface to be sanitized, for example, an accessible surface of a LAV. Thus, in some preferred embodiments, a sanitizing element is disposed in a pre-fabricated shell or housing, either during the manufacturing process or even in the field, where a sanitizing element is inserted or otherwise associated with a suitable shell, housing, or other container designed to accept a particular sanitizing element.

Turning to embodiments wherein the shell is pre-fabricated, the shell can be produced using any suitable process (e.g., casting, extrusion, molding, and a forming process such as pressure-forming, thermoforming, and vacuum-forming) using any suitable material, or combination of materials, although materials amenable to various molding or forming processes are preferred. Representative materials include any suitable plastic or polymer, particularly medical grade plastics and urethanes. Laminates made of two, three, or more layers of suitable materials can also be employed for shell fabrication. Preferred processes injection molding and forming processes (e.g., pressure-, heat-, and vacuum-forming) designed for use with thermoplastics.

A thermoplastic is a material that is plastic or deformable, melts to a liquid when heated and freezes to a brittle, glassy state when cooled sufficiently. Most thermoplastics are high molecular weight polymers whose chains associate through weak van der Weals forces (polyethylene); stronger dipole-dipole interactions and hydrogen bonding (nylon); or even stacking of aromatic rings (polystyrene). Many thermoplastic materials are addition polymers. These include vinyl chain-growth polymers such as polyethylene and polypropylene. Other thermoplastic polymers include acrylonitrile butadiene styrene, polyacrylates, polyacrylonitrile, polycarbonate, polyamides (including naturally and synthetic polyamide materials, e.g., nylons, aramids, etc.), polyester, polystyrene, polysulfone, polyvinyl chloride, cellulose acetate, ethylene-vinyl acetate (EVA), and fluoroplastics (including polytetrafluoroethylenes).

Thermoplastic polymers differ from thermosetting polymers in that the former can, unlike the latter, be remelted and remolded. Thermosetting plastics (thermosets) can also be used to make shells, and are polymer materials that are formed into desired shapes by curing, generally by heating, irradiation, or chemical reactions, to a stronger form that cannot be melted and re-shaped after curing. They are usually liquid or malleable prior to curing, and designed to be molded into their final form, or used as adhesives. Curing transforms the resin into a plastic or rubber by cross-linking of chemically active sites in the polymers, linking them into a rigid, solid three-dimensional structure. Thermosets are generally stronger than thermoplastics due to chemical cross-linking between polymer chains. Thermosets include vulcanized rubber, bakelite (a phenol formaldehyde resin), melamine resin, polyester resin (used in glass-reinforced plastics/fiberglass), and epoxy resin (used as an adhesive and in fiber-reinforced plastics).

Thermoplastic and thermoset materials can be shaped using any suitable process, including reactive injection molding, extrusion molding, compression molding, blow molding, thermoforming, vacuum-forming, and spin casting. If necessary, the resulting parts may be machined or otherwise treated, for example, with a coating, after manufacture.

Other materials suitable for forming pre-fabricated shells or housings are thermoplastic elastomers. These materials are a class of copolymers or a physical mix of polymers (usually a plastic and a rubber) having both thermoplastic and elastomeric properties. While most elastomers are thermosets, thermoplastics are in contrast relatively easy to use in manufacturing, for example, by injection molding. Thermoplastic elastomers have features typical of rubbery materials and plastic materials. For example, they are elastic; however, unlike thermoplastics, they can not be remelted and remolded.

As already described, the shells or housings used in the invention can also be made from combinations of materials. For example, housings can be made from materials comprising two, three, or more layers. The layers may be coextruded or laminated, after which they can be formed (e.g., via pressure forming, thermoforming, or vacuum-forming) into the desired housing shape. As a representative, currently preferred example, housings can be made from a multi-layer structure that includes a cyclic olefin copolymer (COC) layer. Such housings are deformable and clear or translucent. Cyclic olefin copolymer (COC) is an amorphous polymer that has a transparency similar to glass and also has a high moisture barrier with a low absorption rate. As such COCs are also excellent vapor barriers. COCs are used in consumer applications including food and pharmaceutical packaging. Commercially available COC structures used in blister packs are typically coextruded as COC core between thin outer layers. Outer layers (e.g., polypropylene, polyvinyl chloride, polyvinylidene chloride-coated polyvinyl chloride, etc.) can also be placed on a COC core via lamination. Housings made from such materials can be clear, transparent, or translucent.

With regard to preferred embodiments wherein a sanitizing element is disposed within a grippable housing or shell, the housing typically contains an open cavity that defines a cleaning port adapted to receive a sanitizing element and engage an access point of a LAV, e.g., a catheter hub or similar article. Such a cavity is typically defined by an opening that allows a portion of the sanitizing element to be brought into contact with a LAV access port, a bottom disposed opposite the opening and upon which the sanitizing element is positioned, and at least one wall, the upper portion of which defines the opening and a lower portion of which adjoins the bottom. The cavity may be of any suitable size and shape, with the understanding that the particular configuration (i.e., size and shape) of the cavity preferably takes into account the configuration of the access point, e.g., catheter hub of a luer access valve, with which the sanitizing unit is designed to be engaged.

In preferred embodiments, the bottom of the cavity comprises a seat against which the sanitizing element is disposed. In some embodiments, such a seat comprises a substantially planar surface, whereas in others, the seat may comprise two or more portions positioned differently in relation to each other. For example, in some particularly preferred embodiments, a circular seat will comprise a substantially planar outer ring portion and an inner portion that protrudes above the outer ring portion when viewed from the side. The protruding, or raised, inner portion can have any desired shape, and can even be configured to contain a flange element elevated above the plane defining the upper surface of the seat's outer ring portion that can engage the sanitizing element and help to retain it.

As already described, the cavity of a cleaning unit can be of any suitable configuration. Cylindrical bore shapes of any desired width and depth are particularly preferred. Indeed, in some of these embodiments, the cylindrical bore can be configured to mate with a threaded portion, particularly when the access point to be cleaned is a threaded catheter hub (e.g., as used for intravenous lines, central venous lines). Examples of such threaded hubs include those that employ a luer lock connector. In other preferred embodiments, the cavity does not contain complementary surface features on the wall(s) of the bore designed to specifically mate with a threaded catheter hub, but is wide enough to so that the at the sanitizing element can be brought into contact with the threaded portion of the catheter hub when the sanitizing unit is brought into contact with the needleless valve.

Preferably, the outer surface of a shell has a non-slip surface, i.e., one having a high coefficient of friction so that when the capping/sanitizing article is held in a user's hand and positioned to sanitize a LAV, it can be manipulated, for example, using a twisting or rotating motion, with minimal or no slippage in the user's bare or gloved hand. Examples of such surfaces include those having ridges, valleys, dimples, bumps, or other features designed to enhance friction, as well as combinations of two or more of such features. Such features can be introduced into the housing surface as part of the manufacturing process, and if desired in a particular application, materials having high grip levels can also be used to produce shells. Alternatively, a non-slip coating can be applied to at least the grippable portion of a housing. Also, as already described, thin, flexible, and deformable shells and housings can be manufactured from suitable materials, or combinations of materials. In such embodiments the housing of such devices, when gripped by user, for example, when engaging a LAV, can deform under the gripping pressure applied by the user to better engage the surface of the valve being sanitized and/or, in some embodiments, to cause release of some portion of the liquid sanitizing reagent from the sanitizing element. At the same time, through her/his fingers the user can gain tactile feedback as to the sanitizing component/LAV engagement as the article is rotated or otherwise moved by the user in relation to the valve.

The articles of the instant invention also include at least a reservoir capping component configured for tight-fitting association to the discharge port (typically a luer connector) of a medical fluid reservoir, for example, a fluid-filled syringe, an IV bag, etc. The capping component can be manufactured as a separate component designed to be mated with a complementary feature on the housing of the sanitizing component. Alternatively, the capping and sanitizing components can be manufactured as an integrated unitary structure. For example, one embodiment of a two-piece article, the housing of the sanitizing component includes a structure, for example, a tapered or threaded post, that can be used to securely but detachably mate the sanitizing component to complementary features on the reservoir capping component. Given the prevalence of LAV use in hospitals, a particularly preferred embodiment involves incorporating into the shell of the sanitizing component a post structure that sufficiently mimics or replicates the threaded portion of the valve stem of a LAV. The housing of the capping reservoir is manufactured to contain, preferably opposite the reservoir capping structure, a LAV capping component that is designed to receive and securely mate to the threaded portion of the valve stem of a LAV, typically through a complementary threaded portion. In this way, the sanitizing component can be securely attached to the capping component via the LAV-mimicking post structure of the sanitizing component and the receiving structure of the capping component. After a LAV has been sanitized using the sanitizing component (which may or may not first be disconnected from the reservoir cap) and fluid has been injected into the patient via the LAV, the LAV can, if desire, be capped using the LAV-capping component of the component.

In many preferred embodiments, a multi-purpose capping/sanitizing article of the invention also includes one or two seals secured to the housing or shell so as to cover at least the sanitizing region of the sanitizing element disposed in the sanitizing component. Sealing of the sanitizing component can prevent tampering, mass transfer, and long-term stability. In embodiments where the multi-purpose capping/sanitizing element is not secured to a medical fluid reservoir during the manufacturing/assembly process, the reservoir capping component of the article will also typically be sealed to as to maintain sterility of the internal surfaces of the capping component until just prior to its attachment to the discharge port of a medical fluid reservoir in order to cap it.

In any event, a suitable seal can be formed from any suitable material and can be attached to the shell using any suitable process. Preferably, the seal is formed from an impermeable material so as to prevent mass transfer (e.g., gas exchange, evaporation of a liquid sanitizing reagent from the sanitizing element, etc.) between the exterior environment and the interior of the sanitizing component. Suitable seal materials include foils and plastics and multi-layer materials. Depending on the seal material chosen, it is attached to the shell or housing a suitable process. For example, the seal may be adhered to the shell using an adhesive or other bonding agent that is biocompatible and also compatible with the materials used to form the sanitizing element and the shell or coating of the article.

One preferred sealing method is heat-sealing, preferably induction sealing. Induction sealing is a non-contact method of heating a metallic disk to hermetically seal the top of plastic or glass containers. The sealing process takes place after the sanitizing element has been placed, for example, into the cavity of a suitable plastic shell or housing. In such a method, the foil seal comprises a thin conductive metallic foil (e.g., aluminum foil) having a polymer film laminated to one surface of the foil. The seal is positioned over the opening in the housing. Once positioned, the seal is pressed down onto the lip of shell by the sealing head, the induction cycle is activated, and the seal is bonded to the shell. The induction cycle typically involves passing the seal and shell assembly under a sealing head having an induction coil, which emits a varying electromagnetic field. As the assembly passes under sealing head the conductive foil is heated. In a matter of seconds this heating causes the polymer film of the seal to heat and flow onto the lip of the shell. When cooled, the polymer creates a bond with the shell, resulting in a hermetically sealed assembly. Neither the shell nor the sanitizing element is affected. Such processes can be performed using a hand held unit or, for large-scale production, using an automated production line. In production line formats, the foil is typically provided in a reel, and an automated system is used to die cut and position individual foil seals with the sanitizing and reservoir capping components to be sealed. In any event, the particular sealing conditions and equipment used will depend on such factors as the number of units to be manufactured, the particular configuration of the shell, the chemical compositions of the shell and sealing material, and the components of the sanitizing element. Conduction sealing another, albeit less preferred, heat sealing method that can also be used.

A seal can also be welded to the shell. An example of such a process is ultrasonic welding, whereby high-frequency ultrasonic acoustic vibrations are used to weld objects together, usually plastics, particularly molded thermoplastics, and especially for joining dissimilar materials.

The type of seal used will determine how it is to be removed, if at all. For example, in some embodiments, the seal is designed to be separated from the shell (or sanitizing element, if no shell is employed in the particular device), for example, by pealing, by a health care worker immediately prior to use in order to expose the sanitizing element prior to bringing it into contact with a LAV to be sanitized. In other embodiments, the seal may contain perforations or be scored or otherwise pre-fatigued so that the seal can easily be punctured in order to gain access to the sanitizing element disposed in the shell or housing, for example, by pressing a sanitizing element according to the invention that further comprises a puncturable seal against a LAV to be sanitized.

In general, the multi-purpose capping/sanitizing articles of the invention are provided to users in a sealed, sterile manner. Typically this involves securing a seal to the shell to cover the exposed access port openings. After sealing, the articles of the invention are preferably packaged into a suitable container, for example, a foil pouch, for storage and transport. If desired, labeling information, logos, artwork, manufacturing and regulatory data (e.g., lot number, expiration or “use by” date, etc.) may also be printed or otherwise applied to individual articles. In addition, information such as a bar code (to allow use of the device to tracked) may also be included on individual articles. In particularly preferred embodiments, packaged multi-use articles according to the invention are sterilized using a suitable process, such as irradiation. As will be appreciated, articles may be packaged individually or in groups of two or more units as kits, which can further include instructions for use of the capping/sanitizing article(s).

In a particularly preferred practice, the multi-purpose capping/sanitizing articles are sterilized as part of the manufacturing process. Here, “sterilization” refers to any process that effectively kills or eliminates transmissible agents, e.g., bacteria, viruses, fungi, prions, spores, etc. that may be present in any component of a device according to the invention. In preferred embodiments, sterilization can be achieved by heating, chemical treatment, irradiation, and other processes. Indeed, any sterilization process compatible with the materials used to make the article can be employed. A particularly preferred sterilization process is an irradiation process. Such processes include irradiation with x-rays, gamma rays, or subatomic particles (e.g., an electron beam). In general, when a sterilization process is used in the context of the invention, the process is employed on a capping/sanitizing article after it has been sealed and/or packaged.

As those in the art will appreciate, when such articles (or others according to the invention) are manufactured, the capping and sanitizing components can each be covered by a removable seal, after which the assembled article can be packaged and sterilized, if desired. If appropriate, articles can be packaged separately in individual foil or plastic pouches, after which they may be packaged in bulk into larger containers (e.g., boxes or bags). They can then be sold directly to health care providers or to manufacturers of fluid reservoirs, such as manufacturers of IV sets, fluid-filled syringes, and the like.

The invention also concerns methods of using the instant multi-purpose capping/sanitizing articles. Such methods include using the articles to cap medical fluid reservoirs (e.g., fluid-filled syringes, IV sets, etc.) and to sanitize luer access valves to which such medical fluid reservoirs are to be connected just prior to making such connection. In certain preferred embodiments, the articles of the invention also then allow a just-accessed LAV to be capped until further use. Capping in this way can serve to reduce exposure of fluid path portions of the LAV to pathogens and infectious agents present in the environment that might otherwise be able to establish themselves on the exposed LAV surface(s).

To perform such methods, the sanitizing region of the sanitizing component of a capping/sanitizing article of the invention is contacted with the surface of the LAV to be sanitized, typically just before it is to be connected to a fluid-containing medical reservoir (e.g., an IV bag, syringe, etc.) that contains a solution to be delivered to a patient. In preferred practice, once in contact with the LAV, the article is moved in relation to the valve, for example, by rotation or twisting. When the article is one that employs housing that is deformable under gripping pressure applied by a user, the user will gain tactile feedback regarding the article-valve interaction through her/his fingers as the article is rotated or twisted. Such contact and sanitizing action can be for any desired period, with periods of about one second to about ten to twenty seconds being particularly preferred. After contact, the article is removed from the valve, after which, for example, the fluid-containing medical reservoir is connected to the fitting. In preferred embodiments where the sanitizing reagent is a solution, the surface(s) of the fitting contacted with the sanitizing element are dried, either by evaporation or through contact with a sterile, dry, highly absorbent material prior to connection with the LAV, particularly when the article employs a liquid sanitizing reagent, some of which will likely be released from the sanitizing element when it is brought into contact with the valve to be sanitized. It will be appreciated that the articles of the invention can be used manually. In those embodiments where the fluid-containing medical reservoir is connected to the valve is a syringe that is removed following introduction of fluid into the patient through the LAV and wherein the capping/sanitizing article used includes a LAV-capping component on the capping portion of the device, the LAV-capping component can be used to cap the LAV, if desired.

### REPRESENTATIVE EMBODIMENTS

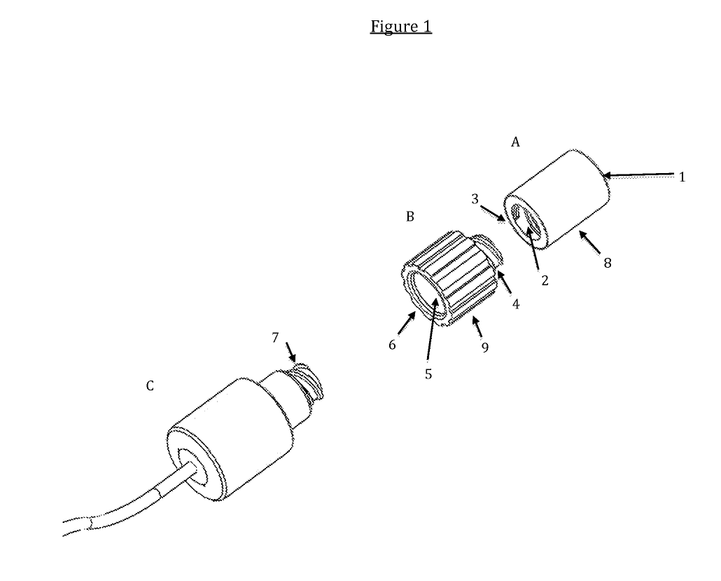

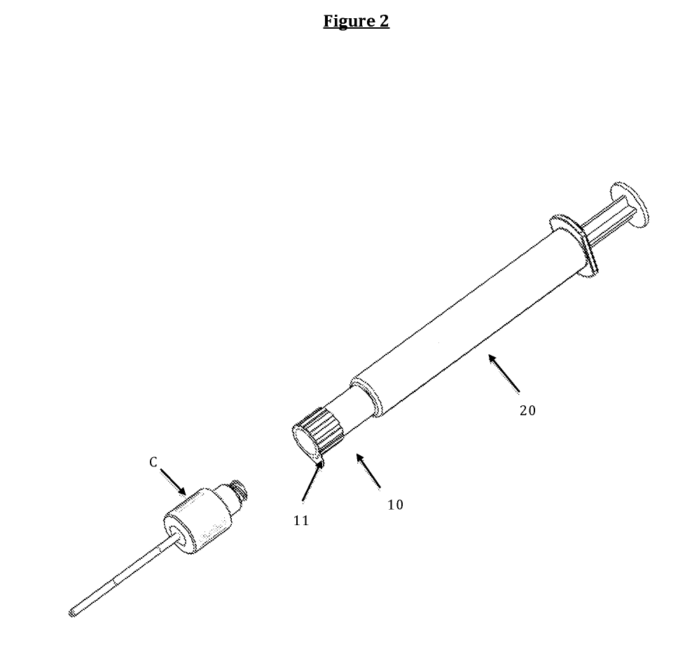

The following descriptions concern several representative embodiments of the invention, which are described in FIGS. 1 and 2. The embodiments described herein merely illustrate a subset of embodiments encompassed by the invention bounded by the appended claims.

FIG. 1 shows a preferred 2-piece embodiment of the invention. As shown in this figure, the capping/sanitizing article of the invention comprises two pieces, A and B. Also shown is a luer access valve, C. In the figure pieces A and B are shown disconnected from each other. Piece A is the reservoir cap, while piece B is the sanitizing component, which includes a sanitizing element (5) disposed in a cavity in the housing (9) that can be accessed through an access port (6). In this embodiment, reservoir cap A attaches to a fluid-filled syringe (not shown) via a bore (1) in the reservoir cap accessed via the open end of the bore (1). The bore is configured to tightly engage the complementary “LUER LOK” receptacle commonly found on medical syringes, Such engagement can be any suitable mechanical engagement, including press-fitting between complementary pieces, threaded engagement between complementary threaded portions, etc. The bore is closed at the end opposite the opening that allows access to the bore.

The housing (8) of reservoir cap A also includes a LAV cap component (3) opposite the end of the reservoir cap configured to mate to a fluid reservoir. The LAV cap (3) includes an opening in a second bore (2), which in this embodiment is threaded so as to engage complementary threads on the post (4) on the sanitizing component (B). The threads in the second bore (2) allow the pieces A and B to be easily connected and disconnected. In this embodiment, the threads in the second bore (2) also allow the cap (piece B) to be readily connected to the threaded valve stem (7) of the LAV shown as part C. Accordingly, such an article can be used initially to cap fluid reservoir such as fluid-filled syringe or IV bag for delivery of medication, nutrients, etc. through a LAV. Just prior to delivering the fluid through the LAV the sanitizing component (piece B) can be brought into contact with the threaded portion (7) of the LAV (piece C). After sanitizing the LAV the capping/sanitizing article (A+B) can be removed from the fluid reservoir, which is then connected to the LAV. When the reservoir is a syringe, after delivering the contents of the syringe through the LAV it is removed. The LAV can then be immediately capped using the LAV cap portion of the cap component (piece A), which is readied for use by disconnecting it (in this embodiment, by unscrewing it) from the sanitizing component (piece B) followed by attaching it to the LAV (in this embodiment, by screwing it onto the threaded portion (7) of the LAV). Those in the art will appreciate that the capping and sanitizing components can be used in different sequences, as well.

FIG. 2 depicts an embodiment of the invention wherein the capping/sanitizing article (10) is attached to a fluid-filled syringe (20). As shown, the sanitizing component still retains its removable seal (11). Also shown is a luer access valve (C). The capping/sanitizing article can be a unitary structure in which the capping and sanitizing components are integrated into a single device having a fluid reservoir capping function at one end and a sanitizing component at the other end. This can be accomplished, for example, by manufacturing a single housing that contains two cylindrical cavities separated by a common shared wall. Each cylindrical bore is open at the outer end of the housing. One bore is configured as the reservoir cap, and thus is adapted to tightly engage the fluid delivery portion (i.e., discharge port such as a LUER LOK receptacle on a syringe). When engagement is of a press-fit type, the bore of the cap component typically is sized to have an internal diameter the same size as or slightly larger than the outer diameter of the LUER LOK receptacle so that the two parts, when brought together, tightly engage, preferably to form a watertight seal. If desired, for extra strength and tight sealing the cap component may be configured such that the both the inner and outer surfaces of the hub of a LUER LOK receptacle can be engaged. This can be accomplished by including a hollow inner cylinder positioned inside the cap component bore that is spaced from the inner wall of the bore so as to accommodate the thickness of the LUER LOK receptacle hub. The inner cylinder is hollow so as to accommodate the luer taper (containing the discharge port) extending from the central portion of the LUER LOK receptacle. If desired, the hollow portion of the inner cylinder can be of a shape and size to matingly engage the luer taper over at least a portion of its length.

If a threaded, screw-type engagement is used, those in the art will appreciate that both the inner and outer surfaces of the hub of a LUER LOK receptacle will preferably be engaged by the cap component. This can be accomplished, for example, by including a hollow inner cylinder positioned inside the cap component bore that is spaced from the inner wall of the bore so as to accommodate the thickness of the LUER LOK receptacle hub. The inner cylinder is hollow so as to accommodate the luer taper (containing the discharge port) extending from the central portion of the LUER LOK receptacle. If desired, the hollow portion of the inner cylinder can be of a shape and size to matingly engage the luer taper over at least a portion of its length. The outer surface of the inner cylinder will include threads configured to engage complementary threads in the inner surface of the LUER LOK receptacle hub.

As an alternative to unitary capping/sanitizing articles, the capping and sanitizing components can be separately manufactured devices adapted to be easily joined and disconnected (for example, by press-fitting or by being screwed together) at some point during the manufacturing process. One such embodiment is depicted in FIG. 1, and has already been described above. After manufacture the devices can be joined about their complementary mating structures. For example, a threaded post on one component and a suitably sized bore with complementary threads in the other component can be used. Similarly, a gradually tapered post on one component and a complementary bore in the other component sized and shaped to receive the post upon press fitting can be used. Other suitable structures for secure mechanical fitment of two parts are well known in the art and can be readily adapted for use in practicing this invention.

As described in the context of a unitary device, when the capping and sanitizing components are separate devices, the capping component preferably includes both a fluid reservoir cap and a LAV cap, typically disposed at opposite ends of the cap component. In these embodiments, the fluid reservoir cap will be designed using the same considerations as taken into account when designing a unitary capping/sanitizing device, and the structures used can be similar or the same.

Turning to the LAV cap portion included in some preferred embodiments, it typically consists of a cylindrical bore open at one end and closed at the other, often via a bottom wall or like structure shared with the bottom of the bore of the fluid reservoir cap at the other end of the cap component. The bore of the LAV cap portion is preferably sized and shaped to receive the threaded valve portion of a LAV. As such, the threaded post of the sanitizing component intended for connection to the cap component via the LAV cap to form a capping/sanitizing article of the invention is likewise manufactured to replicate the size and shape of the threaded valve portion of a LAV.

All of the compositions, articles, and methods described and claimed herein can be made and executed without undue experimentation in light of the present disclosure. While the, articles and methods of this invention have been described in terms of preferred embodiments, it will be apparent to those of skill in the art that variations may be applied to the articles, methods, and compositions without departing from the spirit and scope of the invention. All such variations and equivalents apparent to those skilled in the art, whether now existing or later developed, are deemed to be within the spirit and scope of the invention as defined by the appended claims.

All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A patentable multi-purpose article configured to sanitize a luer access valve and to cap a fluid reservoir, optionally a fluid-filled syringe or IV set, comprising:
a. a sanitizing component that includes (i) a sanitizing element comprising a substrate and a sanitizing reagent dispersed in the substrate prior to use, wherein the substrate has a sanitizing region capable of engaging an accessible surface of a luer access valve, and (ii) a shell disposed about the substrate and having an access port that allows the sanitizing region of the substrate to be brought into contact with and sanitize an accessible surface of a luer access valve; and
b. connected to the sanitizing component a reservoir cap configured for attachment to a fluid-dispensing end of a fluid reservoir, optionally a fluid-filled syringe or IV set.

2. An article according to claim 1 further comprising a seal secured to the shell and covering the access port of the shell.

3. An article according to claim 1 wherein the sanitizing component and reservoir cap are integrated in a unitary structure.

4. An article according to claim 1 wherein the sanitizing component can be removably connected from the reservoir cap.

5. An article according to claim 4 wherein the reservoir cap further comprises a luer access valve cap.

6. An article according to claim 1 further comprising a fluid reservoir, optionally a fluid-filled syringe, removably connected to the article via the reservoir cap.

7. An article according to claim 1 that is sterile.

8. A kit comprising an article according to claim 1 packaged in a single-use container.

9. A method for sanitizing a luer access valve, comprising contacting a luer access valve with a sanitizing component of an article according to claim 1 in a manner sufficient to sanitize the luer access valve.

10. A method for delivering a fluid to patient, comprising:
a. performing a method according to claim 9, wherein the sanitizing article is connected to a fluid reservoir, optionally a fluid-filled syringe, via the reservoir cap; and
b. removing the reservoir cap, connecting the fluid reservoir to the luer access device, and delivering fluid from the fluid reservoir to the patient.

11. A method for capping a luer access device, comprising:
a. performing a method according to claim 9, wherein the sanitizing article is connected to a fluid reservoir, optionally a fluid-filled syringe, via the reservoir cap, and wherein the sanitizing component can be removably connected from the reservoir cap to reveal a luer access valve cap;
b. removing the reservoir cap, connecting the fluid reservoir to the luer access device, and delivering fluid from the fluid reservoir to the patient;
c. disconnecting the fluid reservoir from the luer access device; and
d. placing the luer access valve cap on the luer access valve.
